(19) United States
(12) Patent Application Publication
(10) Pub. No.: US 2021/0205016 A1
(43) Pub. Date: Jul. 8, 2021

(54) DEVICE FOR TREATMENT BY PULSED LASER EMISSION

(71) Applicant: EUROFEEDBACK, EVRY CEDEX (FR)

(72) Inventor: Georges SAFRAOUI, VILLEJUST (FR)

(21) Appl. No.: 17/057,581

(22) PCT Filed: May 22, 2019
(86) PCT No.: PCT/EP2019/063257

§ 371 Date: Jan. 13, 2021

(30) Foreign Application Priority Data
May 22, 2018 (FR) 1854251

(51) Int. Cl. A61B 18/20; A61N 5/06

(52) U.S. Cl. CPC A61B 18/203; A61B 2018/00476; A61N 5/062; A61N 5/0617

(57) ABSTRACT

A device for treating, in particular for hair removal or photo-rejuvenation or vascular treatment, a part of a human body by emitting light pulses. The device includes a handpiece for applying the light pulses to the part of the body. The handpiece is able to move in relation to the body. The handpiece includes a window for outputting the light pulses onto a localized area of the part of the body, and an optical, capacitive or inertial motion detector. The device includes a control system for controlling the emission of the light pulses, configured so as to automatically trigger the light pulses during the movement of the handpiece at a rate determined on the basis at least of at least one item of information provided by the motion detector.

The present invention relates to devices for treating a part of a human body by emitting light pulses, comprising a handpiece connected to a base station, in particular those called LPP (polychromatic pulsed lamp) or IPL (intense pulse light) and those with laser radiation, and to corresponding treatment methods.

The invention preferably relates to treatments such as photo-rejuvenation, hair removal or vascular treatment.

Treatments of this type are being increasingly practiced by professionals by way of devices that have become very widespread in recent years and have taken a very important place in medical and esthetic practices.

These devices usually comprise a handpiece for delivering light pulses to the part of the body to be treated. The handpiece has a light pulse emission window that does not allow a large part of the body to be treated with a single pulse. The treatment is therefore usually performed by an operator who manually triggers a light pulse on an area of the part of the body to be treated and moves the handpiece in order to then trigger a new light pulse on a new area adjacent to the previous one, and so on. Such a method is relatively slow and expensive.

International application WO 01/26573 discloses a device for treating the skin, in particular for hair removal, by emitting laser pulses onto the skin, comprising an electronic system determining the position of the handpiece on the skin and automatically triggering the emission of a light pulse when the area on which the handpiece is arranged is to be treated. In this device, the handpiece comprises a motion detector for detecting the movements of the handpiece in the form of a roller and a position detector for detecting the position of the handpiece in relation to a fixed element in the form of a transponder and one or more detectors. The motion detector in the form of a roller is able to detect the movements of the handpiece only in one direction. In addition, such a roller is not compatible with the application of a gel to the skin, which would no longer allow the roller to roll over the skin without sliding. If the body to be treated moves, the information regarding the areas still to be treated may be distorted and some areas may not be treated.

Application US 2013/0237973 also discloses a hair removal device that works by emitting laser pulses onto the skin, comprising a control unit that distinguishes between areas that have received laser emission and areas that have not received any on the basis of information on the direction and distance of movement of the handpiece, obtained by way of a detector on the handpiece, coupled or not coupled to a marking on the skin and an external camera coupled to a reference point on the handpiece. The control unit displays this information on a display unit so that the operator is able to identify areas subjected or not yet subjected to hair removal, and move the handpiece and actuate the light pulses accordingly. Various types of detector are described. This device requires the operator to manually trigger each light pulse.

There is therefore a need to benefit from a safe, reliable, precise and fast device that allows a treatment, in particular a photo-rejuvenation or hair removal treatment, or a vascular treatment.

### SUMMARY

The invention meets this need, according to a first of its aspects, using a device for treating a part of a human body by emitting light pulses, comprising:

a handpiece for applying light pulses to the part of the body, the handpiece being able to move in relation to the body and comprising:

a window for outputting the light pulses onto a localized area of the part of the body, and

an optical, capacitive or inertial motion detector,

a control system for controlling the emission of the light pulses, configured so as to automatically trigger the light pulses during the movement of the handpiece at a rate determined on the basis at least of at least one item of information provided by the motion detector.

Such a device allows rapid treatment of the part of the body in that the operator does not have to position the handpiece and then trigger each light pulse. He only has to scan the part of the body to be treated with the handpiece, and the light pulses are generated automatically by the device.

Such a motion detector is advantageous because it makes it possible to determine various information, as will become apparent below.

The device may comprise one or any combination of the following features:

The treatment is non-invasive. The treatment is preferably non-surgical.

The treatment is esthetic.

The part of the body to be treated is an area of the surface of the skin.

The device may comprise a system for the operator to set various treatment parameters, in particular the number of light pulses emitted per pass of the handpiece, the number of passes over each unit area of the part of the body to be treated, as well as at least one parameter relating to at least one feature of the part of the body, in particular the phototype of the skin and/or the features of the hairs to be treated.

The motion detector makes it possible to determine the direction and the speed of movement of the handpiece over the part of the body, preferably the direction and the speed of movement of the handpiece over the skin at least in two dimensions.

The rate is determined such that the distance covered by the handpiece between two light pulses is less than or equal to d, preferably less than d, where d is the dimension of the output window in the direction of movement of the handpiece. The rate is preferably determined so that the distance covered by the handpiece between two light pulses is non-zero, in particular greater than or equal to 10% of d, better still greater than or equal to 25% of d. The distance covered by the handpiece between two successive light pulses is preferably substantially equal to d/N, where N is the number of flashes emitted per unit area in one pass, N being chosen by the operator prior to the treatment. “Number of flashes per unit area in one pass” is understood to mean that a unit area of the part of the body to be treated has received on average a number of flashes N during the time for which it remained under the light pulse output window without interruption. N may or may not be an integer, and N is preferably between 1 and 10, better still between 1 and 5. For example, the distance covered by the handpiece between two successive light pulses is substantially equal to ¼, ⅓, preferably ½ of the dimension of the output window in the direction of movement of the handpiece, or greater than or equal to 80%, better still 90% of the dimension of the output window in the direction of movement of the handpiece.

The rate is determined during the treatment on the basis of the direction and the speed of movement of the handpiece over the skin. If the operator moves the handpiece slowly, the rate will be lower than if he moves his hand quickly.

The maximum emission frequency of the light pulses is greater than or equal to 10 Hz, better still greater than or equal to 30 Hz, even better still greater than or equal to 50 Hz. Such an emission frequency of the light pulses allows rapid and effective treatment of the part of the body at relatively high speeds of movement of the handpiece.

The device may be designed to emit an alert signal when the speed of movement of the handpiece over the skin is too high to allow triggering of the light pulses at the rate that allows the treatment. The alert signal may be a light signal, a message on a viewing system or an audible signal.

The light pulses may be formed by polychromatic pulsed light, in particular intense pulsed light, and preferably laser pulses.

If the light pulses are laser pulses, the device preferably comprises a base station comprising a laser source emitting the laser pulses, the handpiece being optically connected to the laser source by at least one optical guide. Such a configuration allows the light source to be arranged remotely from the handpiece, thereby making the handpiece lighter, and making it possible to overcome the constraints in terms of size or weight of the cooling system for cooling the light source, thereby making it possible to have a cooling system that is more efficient than if it were in the handpiece. The optical guide may be an optical fiber or a liquid optical guide. The laser source may comprise at least one laser diode. The light power emitted by the laser source is preferably between 500 W and 10 000 W.

The laser source preferably comprises a plurality of laser diodes. The diodes are preferably arranged in a plurality of rows. The diodes in one and the same row are for example connected in series and the various rows of diodes are connected in parallel or controlled independently of one another. Each row of diodes preferably has diodes emitting at a wavelength in the infrared band, in particular wavelengths of 750 nm, or of around 1064 nm, and optionally one or more diodes emitting in the visible. All of the laser diodes in the same row, except for any laser diode emitting in the visible, preferably have one and the same wavelength, in particular 750 nm or 1064 nm. The laser diode or diodes emitting in the visible preferably emit at a wavelength that is not filtered by the device and by the glasses worn by the operator, for example in the green. Such diodes allow the light pulses emitted by the handpiece to be visible to the operator, thereby improving safety.

The control system may determine the trigger sequence of the laser diodes, in particular the rows of laser diodes to be triggered, the intensities of each of the rows of laser diodes, the trigger duration of each of the rows of laser diodes, and/or the trigger time of each row of laser diodes, on the basis of at least one feature of the skin, in particular the phototype of the skin, the feature of the skin being able to be determined from the information provided by the motion detector and/or from data entered by the operator. The base station may comprise an optical system between the diode or diodes and the optical guide in order to concentrate the light from the diode or diodes in the optical guide. The optical system preferably comprises a lens, in particular formed of a plurality of microlenses produced on a support, configured so as to concentrate the light from the laser diode or diodes toward the optical guide.

The device, in particular the base station, comprises a cooling system for cooling a light source emitting the light pulses.

The motion detector is arranged on the contact face of the handpiece for contacting the skin.

The motion detector is outside the light pulse output window, preferably adjacent to the light pulse output window. The motion detector is preferably separate from the light pulse output window.

The motion detector detects the movements of the handpiece in at least two different directions, in particular two directions that are not parallel to one another, and are preferably perpendicular to one another.

The motion detector is optical and preferably comprises a light source, in particular an LED or preferably a laser diode, and a multi-pixel optical detector, preferably a camera, configured so as to image a portion of the skin illuminated by the light source. The optical detector preferably takes a number of images per second greater than or equal to 1000 images/s, better still greater than or equal to 2000 images/s, even better still greater than or equal to 4000 images/s.

The optical detector preferably takes images of the skin over a duration of less than or equal to 500 μs, better still less than or equal to 300 μs.

The light source may or may not be continuous, for example stroboscopic.

The images are acquired so as to avoid motion blur and to allow processing of the images from which the speed and the direction of movement are able to be extracted.

The motion detector may be similar to those used for optical mice.

The motion detector may also be of multi-pixel capacitive type. Such sensors are known for generating an image of the skin of the finger for reading fingerprints, and are able to deliver an image of the microrelief of the skin, analysis of which makes it possible to determine the speed and the direction of movement.

The motion detector may be optical and configured so as to produce a color image of the skin. In this case, the motion detector may comprise three light sources of three different colors that are triggered in succession, preferably red, green and blue, respectively, and a multi-pixel optical detector as described above. The light sources may be three laser diodes or three LEDs. As a variant, the three light sources are integrated into a single laser diode or a single LED.

The device may comprise a processing system for processing the images provided by the motion detector. The processing system for processing the images provided by the motion detector may be implemented so as to detect hairs in said images. Said processing system may determine the location on the skin and/or the diameter of each detected hair and/or the number of hairs, in absolute terms or per unit area. The device may estimate, after the handpiece scans the part of the body to be treated, from the information provided by the processing system for processing the images provided by the motion detector, the distribution of the hairs on the part of the body to be treated and/or their associated diameters and/or the number of hairs on the part of the body to be treated. The device may make this estimation by extrapolating, over the entire part of the body, the information provided by the processing system for processing the images provided by the motion detector, taken on the areas of the skin that are imaged by the motion detector throughout the treatment. This estimation may be made whether or not the light pulses are triggered. It is possible to pass the handpiece over the part of the body to be treated without any light pulses being emitted, in order to obtain this information prior to the treatment. Such estimations in particular make it possible, over the course of the hair removal sessions, to create a history of the hairiness of the part of the body to be treated in order in particular to evaluate the effectiveness of the treatment.

The processing system for processing the images provided by the motion detector may be designed to determine a feature of the skin, and the control system may drive the light pulses on the basis of the feature of the skin. For example, the processing system determines the phototype, and the light power is selected automatically, or suggested to the user.

The processing system for processing the images provided by the motion detector is able to detect pressing of the handpiece on the skin, the control system being configured so as to prevent a light pulse from being emitted when the handpiece is not being pressed against the skin. This helps to improve operational safety and to guarantee an effective treatment. The pressure that is exerted displaces the blood and the effect of the hair removal treatment is improved. Pressing may be detected by detecting whether the image of the skin as acquired by the motion detector is crisp; if the pressing is insufficient, the image will be blurry.

The device does not have any rotary moving element on the face of the handpiece that comes into contact with the skin,

The motion detector does not have any rotary element,

The handpiece comprises a surface at least partially defining the window and intended to come into contact with the skin,

The light pulse output window is configured so as to allow the light pulses to be emitted only over a single area at a given position of the handpiece.

The window preferably has a rectangular or square cross section.

At the output of the optical guide, the cross section of the beam may be circular; the handpiece may then comprise an optical system arranged upstream of the window, in particular between the optical guide and the window, for transforming the cross section of the beam before the window, in particular at the output of the optical guide, into an emitted beam cross section having, in a direction of movement of the handpiece, two substantially parallel opposing edges, in particular having a rectangular or square shape.

Within the cross section, the light intensity may not be homogeneous, for example being greater in the center than at the edges, for example in accordance with a Gaussian intensity profile; in this case, the optical system may comprise a means for making the intensity more uniform in the cross section. The optical system may in particular comprise a lens comprising a plurality of microlenses formed on a support for modifying the distribution of the beam intensity and a diffuser behind the plurality of microlenses for even further homogenizing the distribution of light over the entire cross section of the beam.

The device may comprise a settable focusing system for varying the cross section of the light pulse at the output of the handpiece. This may make it possible to reduce the size of the treated area or by contrast to enlarge it. The handpiece may comprise a detector configured so as to detect the focal length of the focusing system, in particular an optical, mechanical or magnetic detector. The device may comprise a computer configured so as to adapt at least one feature of the light pulses on the basis at least of an item of information provided by the focusing detection system. The focusing system may comprise a lens and a telescopic portion of the handpiece for changing the distance between the lens and the light pulse output window. The focusing system may comprise a plurality of preset focal lengths associated or not associated with specific light pulse parameters. For example, the telescopic portion of the handpiece may comprise locking elements for blocking the telescopic portion in certain extensions. The locking elements may be reliefs complementary to the various parts of the portion or reliefs or holes cooperating with one or more added parts. The device may then automatically change the light pulse parameters according to the specific light pulse parameters associated with the focus set by the operator. Such settable focusing makes it possible to set the surface dimension of the treated surface and the power of the light pulses on the basis of at least one feature of the area to be treated. For example, if the light pulse is focused on a smaller surface, the intensity is higher than if the pulse is focused on a larger area, with everything else being the same.

As an alternative, the focal length may be set continuously such that any focal length between a minimum focal length and a maximum focal length may be chosen.

The handpiece comprises a metal sole surrounding the window and intended to come into contact with the skin during the application of the handpiece to the skin. Such a window may help to cool the skin.

The handpiece comprises a cooling system for cooling the handpiece. The cooling system for cooling the handpiece preferably comprises a liquid at a temperature of less than 5° C. and at least one Peltier-effect cell and/or at least one heat pipe. The cooling system preferably comprises at least one Peltier-effect cell and at least one heat pipe, the heat pipe being in thermal contact with a metal sole in contact with the skin and with the Peltier-effect cell, the Peltier-effect cell being cooled on its hot side by the coolant.

The cooling system may comprise at least one heat pipe, the heat pipe being in thermal contact with a sole in contact with the skin and with a metal part cooled by a liquid, preferably at a temperature of less than 5° C.

As another variant, the cooling system comprises at least one Peltier-effect cell in contact with a metal sole in contact with the skin and a liquid at a temperature of less than 0° C.

The temperature of the face of the handpiece configured so as to come into contact with the skin has, during operation, a temperature of less than or equal to 20° C., preferably less than or equal to 15° C., better still less than or equal to 10° C., even better still less than or equal to 5° C. The temperature of the handpiece is preferably greater than or equal to −10° C., better still greater than or equal to −5° C. The handpiece is preferably temperature-controlled.

Each unit area of the skin may receive, from the light pulse or pulses, a fluence or energy flux density, per pass of the handpiece, or one accumulated over all of the passes during the treatment, of between 5 J/cm² and 100 J/cm² , preferably between 5 J/cm² and 60 J/cm². “Per pass” is understood to mean that each unit area has received on average the cumulative energy flux density indicated during the time for which it remained under the light pulse output window without interruption. “Accumulated over all of the passes” is understood to mean that each unit area has received on average the cumulative energy flux density indicated during the time for which it remained under the light pulse output window throughout the treatment.

Each light pulse emitted by the output window has a power flux density greater than or equal to 200 W/cm², preferably greater than or equal to 1 kW/cm², better still greater than or equal to 2 kW/cm².

The device comprises at least one camera, preferably at least two cameras, each arranged so as to have the handpiece in its field of view, preferably a field of view that encompasses the entire part of the body to be treated, and a processing system for processing the images provided by the camera or cameras in order to locate the handpiece with respect to the part of the body from at least these images and optionally from the information provided by a determination system for determining the movements of the part of the body, as described below. The processing system for processing the images provided by the camera or cameras preferably locates the handpiece with respect to the part of the body independently of the emission of the light pulses. The camera or cameras are preferably fixed in relation to the part of the body to be treated. The device preferably comprises at least two cameras oriented in different viewing directions with respect to the part of the body to be treated. The handpiece may comprise a marking making it easier for the camera or cameras to recognize. The marking is preferably at a distance of less than or equal to 5 cm, better still less than or equal to 2 cm, even better still less than or equal to 1 cm, from the surface of the handpiece that comes into contact with the skin. The marking is preferably a physical or optical marking, for example a pattern such as a line, an arrow or a cross, a light halo or one or more LEDs on the handpiece. The proximity of the marking on the handpiece to the skin makes it possible to avoid complex calculations in order to deduce the exact location of the treated area from the orientation of the handpiece.

The camera or cameras are preferably arranged on the opposite side of the part of the body from the operator. The risk of said operator obscuring the field of view of the cameras is thus reduced.

The light pulse control system is configured so as to automatically trigger the pulses on the basis at least of the position of the handpiece in relation to the part of the body located by the processing system for processing the images provided by the camera.

The device comprises a temperature detector, in particular a thermal camera or an optical pyrometer, preferably a thermal camera, for monitoring the local temperature of the area exposed to the light pulse. The control system may be configured so as to block triggering of the light pulses or to emit an alert signal for the operator when the local temperature measured by the temperature detector is greater than a limit temperature. The limit temperature is preferably calculated on the basis of the speed of movement of the handpiece and of a predetermined temperature decrease curve, depending on at least one feature of the skin, in particular the phototype of the skin. The limit temperature preferably corresponds to the temperature of the predetermined temperature decrease curve at a time equal to the time between the emission of the light pulse and the measurement of the temperature on the corresponding area. The limit temperature is preferably less than or equal to 60° C., better still less than or equal to 50° C., even better still less than or equal to 45° C. The temperature detector may be fixed during the treatment and image the entire part of the body during the treatment. As a variant, the temperature detector is arranged on the handpiece so as to measure the temperature of the area after it has been subjected to a light pulse. The control system is preferably configured so as to determine the treated areas of the part of the body to be treated using at least one item of information provided by the temperature detector since the start of the treatment and optionally from at least one item of information provided by a determination system for determining the movements of the part of the body, as described below. The handpiece preferably has an orientation on the skin according to a predetermined direction of movement such that the temperature detector is downstream of the output window in the direction of movement of the handpiece over the part of the body to be treated.

The device comprises at least one visual information system for viewing the areas to be treated in relation to the as yet untreated areas of the part of the body to be treated.

The device comprises at least one projection system for projecting at least one item of guidance information onto the part of the body in order to guide the operator during the treatment. The device preferably comprises two projection systems oriented in different viewing directions with respect to the part of the body to be treated. The projection system or systems preferably at least partially illuminate the part of the body to be treated in order to distinguish, in particular through a difference in brightness, color or illumination, between the treated areas and the untreated areas of the part of the body to be treated. The projection system may produce an image on the part of the body that is configured so as to distinguish, in particular through a difference in brightness or color, between the areas of the skin in terms of the number of passes of the handpiece that they have experienced. The illumination by the projection system or systems may be adjusted in real time on the basis of the movements of the body, in particular detected by a determination system for determining the movements of the part of the body, as described below. The guidance information for the part of the body is preferably information relating to the untreated areas of the part of the body to be treated.

The device comprises a treatment viewing system configured so as to display an image of the part of the body, in particular a three-dimensional image, and optionally one or more of the following features:

the position of the handpiece on the body as provided by the camera or cameras,

the treated areas of the part of the body and/or the untreated areas of the part of the body, and/or

the history of the temperature of the skin based on the information provided by the temperature detector since the start of the treatment at each point of the part of the body to be treated.

The viewing system preferably comprises a screen. As a variant, the viewing system is a virtual reality or augmented reality headset displaying information relating to the part of the body to be treated when the operator wearing it is looking in the direction of said part.

The device determines the as yet untreated areas of the part of the body, and the control system is configured so as to automatically trigger the emission of the light pulses when the handpiece is positioned on an as yet untreated area of the part of the body to be treated.

The device comprises a determination system for determining the movements of the part of the body in order to make it possible to take into account the movements of the part of the body during the treatment, in particular in the precise determination of the position of the handpiece and/or the determination of the areas of the body treated by the handpiece. The determination system for determining the movements of the part of the body may comprise a three-dimensional digital model of the human body to be calibrated depending on the body being treated. Such a model makes it possible, on the basis of the information provided by the cameras, to easily calculate the movements of the body without having to analyze the images from the cameras pixel by pixel, these being pre-calculated in the model.

The motion detector is preferably configured so as to detect the movement of the handpiece on the skin in the absence of any particular marking thereon.

As a variant, the movement determination system comprises a visual reference on the skin, for example a boundary of the part of the body to be treated, or better still a marking, in particular a grid on the part of the body to be treated, spaced geometric shapes, in particular distributed regularly over the part of the body to be treated, for example dots, lines, crosses or any other reference marking. The marking may be printed in ink, preferably allowing the wavelength or wavelengths of the light pulses to pass, in particular fluorescent and transparent to infrared. The processing system for processing the images from the camera or cameras may then determine the movements of the body on the basis of the deformations of the grid or of the movements of the shapes with respect to one another that it detects.

As another variant, the determination system for determining the movements of the part of the body comprises projecting fringes onto the part of the body, in particular using the projection system described above or an additional projector, in particular a laser projector, and the camera or cameras described above or an additional camera, in order to analyze the fringes projected onto the skin. Analyzing the deformations of the fringes then makes it possible to deduce the movements of the part of the body.

The determination system for determining the movements of the part of the body preferably analyzes the movements of the part of the body and refreshes the images every t milliseconds, t being less than or equal to 50 ms, better still less than or equal to 20 ms, better still less than or equal to 10 ms.

The device comprises a robotic arm configured so as to move the handpiece over the part of the body, in particular by scanning.

The device comprises a selector for selecting at least one of the following modes:

a manual mode in which each light pulse is triggered manually by the operator, or

an automatic mode wherein the device automatically triggers the light pulses on the basis of the movements of the handpiece.

This allows the operator to choose, in particular for certain small areas, to trigger the light pulses himself. The operator may then dispense with the step of selecting the part of the body, in particular when this is small.

The device may comprise or be connected to a client database for keeping a history of the treatments over the course of the hair removal sessions.

Another subject of the invention, according to this first aspect, is a method for treating a part of the body using the device as described above, comprising the steps of:

applying a gel to the skin of the part of the body to be treated and/or to the handpiece,

applying the handpiece to the part of the body to be treated,

scanning the part of the body to be treated with the handpiece while keeping the handpiece pressed on the skin,

the control system automatically triggering the light pulses when the handpiece moves over the skin.

Applying a gel makes it possible to reduce the index differences between the interfaces passed through by the light pulses, thereby improving the transmission of energy to the skin. The presence of the gel also makes it possible to avoid any release of smoke linked to the destruction of the hairs, thereby making the treatment healthier for the operator and limiting the temperature increase of the skin. The gel applied to the skin is preferably at a temperature of less than or equal to 5° C.

The method may comprise one or a combination of the following features:

The gel is at a temperature of between 2° C. and 5° C. when it is applied.

The method comprises a step of the operator selecting certain parameters, in particular a step of selecting the number of light pulses per pass of the handpiece, the number of passes over each unit area of the part of the body to be treated and/or the phototype of the skin.

During the scanning of the part of the body, the successive passes over one and the same area of the part of the body are spaced by a duration of at least 1 s, preferably at least 2 s, better still at least 3 s.

The method comprises a step of determining the distance that the handpiece has to cover between two successive light pulses, in particular on the basis of the number of light pulses per pass of the handpiece as selected by the operator and of the direction of movement of the handpiece.

The control system automatically triggers the light pulses when the handpiece has covered the determined distance and the area facing the window has not already been treated.

The step of scanning the part of the body with the handpiece may be carried out at variable speed, the control system adapting the rate of triggering of the light pulses on the basis of the scanning speed of the handpiece.

The handpiece scans the part of the body to be treated at a speed of less than or equal to 50 cm/s.

The method may comprise a step of selecting between a plurality of modes, in particular a manual mode in which the operator manually triggers the light pulses or an automatic mode in which the device automatically triggers the pulses depending on the movement of the handpiece over the part of the body. The automatic mode may be triggered by pressing a pedal with a foot.

The method comprises a step of marking the part of the body to be treated, in particular the trace of an outline of the part of the body to be treated directly on the skin or on a viewing system for viewing the treatment, for example using a stylus or a finger or by selecting predetermined areas from views displayed on the viewing system. The marking is preferably made directly on the skin.

The scanning of the handpiece over the part of the body is scanning along adjacent lines while always scanning in the same direction or in opposite directions from one line to another, preferably always scanning in the same direction. As a variant, the scanning may be performed through circular movements or with any other movement.

The handpiece is applied to the skin with a pressure greater than or equal to a predetermined pressure, in particular greater than or equal to 0.1 N/cm², preferably 0.2 N/cm². Such pressure makes it possible to displace the blood under the skin and thus improve the contrast between the skin and the hairs.

The power of the light pulses is modified automatically or manually on the basis of the local temperature of the skin after the emission of a light pulse during the treatment.

The method comprises a step of determining the areas of the part of the body that have not received light pulses during a pass of the handpiece and/or the areas of the part of the body over which the handpiece has passed a number of times less than a predetermined number of passes, in particular defined by the operator prior to the treatment. The method may comprise a step, during the treatment, of signaling the areas that have not received any light pulses during a pass and/or the areas over which the handpiece has passed a number of times less than the predetermined number of passes. The signaling may be performed through a display on a viewing system or through projection onto the body by a projection system for projecting information relating to the progress of the treatment, in particular by distinguishing between fully treated areas, that is to say areas that have experienced the predetermined number of passes, and as yet not fully treated areas, and/or the number of light pulses received on each area per pass and/or the number of passes over each of the areas of the part of the body. Such a display may be performed by using different colors depending on the number of passes experienced by the corresponding area.

The method comprises a step of analyzing the information provided by the motion detector, in particular analyzing the images provided by the multi-pixel optical or multi-pixel capacitive detector by the processing system for processing said images, in order to determine at least one local feature of the skin, in particular its phototype, and a step of adapting the features of the light pulses on the basis of the feature of the skin, in particular its phototype.

The method comprises a step of analyzing the information provided by the motion detector in order to detect the pressing of the handpiece on the skin, in particular by way of a processing system for processing the images provided by the motion detector, a step of interrupting the light pulses and/or a step of signaling to the operator the absence of pressing on the handpiece when the absence of contact between the handpiece and the skin is detected.

The method comprises a step of setting the focal length of the light pulses in order to modify the size of the beam at the output of the handpiece. The focal length of the light pulses may be set by choosing one focal length from among a preset plurality of focal lengths. Each preset focal length may be associated with specific light pulse parameters. The method may comprise a step of automatically modifying the parameters of the light pulse, in particular the power of the light pulses, depending on the specific light pulse parameters associated with the focusing of the set pulse. Such setting makes it possible to set the surface dimension of the treated surface and the power of the light pulses on the basis of at least one feature of the area to be treated.

The method comprises a step of selecting the rows of laser diodes to be triggered, the intensities of each of the rows of diodes and/or the trigger sequence of the rows of diodes on the basis of at least one feature of the skin, in particular the phototype of the skin, the feature of the skin being able to be determined from the information provided by the motion detector and/or from information chosen by the operator.

The method comprises a step of cooling the handpiece and/or the skin in contact with the handpiece. This cooling may be performed by a cooling system integrated into the device, as described above. As a variant, the handpiece and the skin are cooled by an additional cooling device, for example by a device such as a pulsed cold air generator, emitting an air flow in the direction of the area to be treated. The air flow is preferably cooled, in particular to a temperature of less than or equal to −20° C., better still less than or equal to −30° C., even better still less than or equal to −40° C.

The method comprises a step of determining the movements of the part of the body in order to make it possible to take into account the movements of the body during the treatment, in particular in the precise determination of the position of the handpiece and the determination of the areas of the body treated by the handpiece. The part of the body may be scanned automatically by the handpiece using a robotic arm.

The treatment is a hair removal or photo-rejuvenation treatment or a vascular treatment.

In the case of a hair removal treatment, the method may comprise a step of estimating the distribution of the hairs, their associated diameters and/or the number of hairs on the part of the body. This estimation may be made by extrapolating, over the entire part of the body, the information provided by the motion detector, in particular by the processing system for processing the images provided by the motion detector, taken on the areas of the skin that are imaged by the motion detector throughout the treatment. Only part of the skin is imaged when scanning the area to be treated. The percentage of imaged skin is around 10%, preferably 20% or better still 30%.

This step may be performed at the same time as the treatment and/or by scanning the part of the body to be treated with the handpiece before or after the treatment without any light pulses being emitted, in particular by selecting a neutral mode of the device in which the light pulses are not triggered. The method may comprise a step of displaying the distribution of the hairs, their associated diameters and/or the number of hairs on the part of the body on a viewing device.

The method may comprise a plurality of hair removal sessions and a step of estimating the effectiveness of the treatment over several sessions by displaying a history of the hairiness of the part of the body to be treated over all of the hair removal sessions.

The method is a hair removal method and comprises a step of shaving the hairs prior to the treatment, preferably the day before the treatment.

Another subject of the invention, according to a second of its aspects, is a device for treating a part of a human body by emitting light pulses, comprising

a handpiece for applying the light pulses to the part of the body, the handpiece being able to move in relation to the body and comprising a window for outputting the light pulses onto a localized area of the part of the body, and

a temperature detector delivering information representative of the temperature of said area immediately after it leaves the window.

Such a device for treating the part of the body makes it possible to determine the temperature of the part of the body after it has received a light pulse. It also allows the treatment to be traceable and safeguards the operator.

The device may comprise one or a combination of the following features:

The treatment is non-invasive. The treatment is preferably non-surgical.

The treatment is esthetic.

The part of the body to be treated is an area of the surface of the skin.

The handpiece has an orientation on the skin according to a predetermined direction of movement such that the temperature detector is downstream of the output window in the direction of movement of the handpiece over the part of the body to be treated.

The temperature detector is a thermal camera or an optical pyrometer, preferably a thermal camera, for monitoring the local temperature of the area exposed to the light pulse.

The device comprises a control system configured so as to block triggering of the light pulses or to emit an alert signal for the operator when the local temperature measured by the temperature detector is greater than a limit temperature.

The limit temperature may be calculated on the basis of the speed of movement of the handpiece and of a predetermined temperature decrease curve depending on at least one feature of the skin, in particular the phototype of the skin, in particular the limit temperature corresponds to the temperature of the predetermined temperature decrease curve at a time equal to the time between the emission of the light pulse and the measurement of the temperature on the corresponding area.

The limit temperature may be less than or equal to 60° C., better still less than or equal to 50° C., even better still less than or equal to 45° C.

The control system may be configured so as to determine the treated areas of the part of the body to be treated using at least one item of information provided by the temperature detector since the start of the treatment. The treated areas of the part of the body are preferably determined additionally in order to make it possible to take into account the movements of the part of the body during the treatment.

The temperature detector is fixed during the treatment and images the entire part of the body during the treatment.

As a variant, the temperature detector is arranged on the handpiece so as to measure the temperature of the area after it has been subjected to a light pulse.

The handpiece may comprise a motion detector, in particular an optical, capacitive or inertial motion detector.

The device may comprise a control system for controlling the emission of the light pulses, configured so as to automatically trigger the light pulses during the movement of the handpiece at a rate determined on the basis at least of at least one item of information provided by the motion detector.

One or a combination of the features described with reference to the device according to the first aspect of the invention.

Another subject of the invention, according to this second aspect of the invention, is a method for treating a part of a body by emitting light pulses using a device according to this second aspect, comprising the step of monitoring the temperature of the area exposed to a light pulse after the application of said light pulse.

The method may comprise one or a combination of the following features:

The method may comprise a step of applying a gel to the skin of the part of the body to be treated and/or to the handpiece,

The method may comprise a step of applying the handpiece to the part of the body to be treated and a step of scanning the part of the body to be treated with the handpiece, in particular while keeping the handpiece pressed on the skin.

The control system may automatically trigger the light pulses when the handpiece moves over the skin.

One or a combination of the features described with reference to the method according to the first aspect of the invention.

Another subject of the invention, according to a third of its aspects, is a device for treating a part of a human body by emitting light pulses, comprising:

a handpiece for applying the light pulses to the part of the body, the handpiece being able to move in relation to the body and comprising a window for outputting the light pulses onto a localized area of the part of the body, and

at least one projection system for projecting guidance information onto the part of the body in order to guide the operator during the treatment and/or information for assisting with the location of the handpiece and/or the body.

The device may comprise one or a combination of the following features:

The treatment is non-invasive. The treatment is preferably non-surgical.

The treatment is esthetic.

The part of the body to be treated is an area of the surface of the skin.

The light pulse output window is configured so as to allow the light pulses to be emitted only over a single area at a given position of the handpiece,

The guidance information for the part of the body is information relating to the untreated areas of the part of the body to be treated.

The projection system at least partially illuminates the part of the body to be treated in order to distinguish, in particular through a difference in brightness, color or illumination, between the treated areas and the untreated areas of the part of the body to be treated.

The projection system produces an image on the part of the body that is configured so as to distinguish, in particular through a difference in brightness or color, between the areas of the skin in terms of the number of passes of the handpiece that they have experienced.

The projection of the information onto the area of the part of the body by the projection system is adjusted in real time on the basis of the movements of the body.

The device comprises two projection systems oriented in different viewing directions with respect to the part of the body to be treated.

The projection system projects fringes onto the part of the body in order to help determine the movements of the part of the body.

The device comprises a camera for analyzing the fringes projected onto the skin.

The handpiece may comprise a motion detector, in particular an optical, capacitive or inertial motion detector.

The device may comprise a control system for controlling the emission of the light pulses, configured so as to automatically trigger the light pulses during the movement of the handpiece at a rate determined on the basis at least of at least one item of information provided by the motion detector.

One or a combination of the features described with reference to the device according to the first aspect of the invention.

Another subject of the invention, according to this third aspect of the invention, is a method for treating a part of a body by emitting light pulses using a device according to this third aspect, comprising the steps of projecting, onto the part of the body, guidance information or information for assisting with the location of the handpiece with the projection system and of treating the part of the body on the basis of the guidance information and/or of locating the handpiece and/or the body.

The method may comprise one or a combination of the following features:

The method may comprise a step of applying a gel to the skin of the part of the body to be treated and/or to the handpiece,

The method may comprise a step of applying the handpiece to the part of the body to be treated and a step of scanning the part of the body to be treated with the handpiece, in particular while keeping the handpiece pressed on the skin.

The control system may automatically trigger the light pulses when the handpiece moves over the skin.

One or a combination of the features described with reference to the method according to the first aspect of the invention.

Another subject of the invention, according to a fourth of its aspects, is a device for treating a part of a human body by emitting light pulses, comprising:

a handpiece for applying the light pulses to the part of the body, the handpiece being able to move in relation to the body and comprising a window for outputting the laser pulses onto a localized area of the part of the body,

at least one camera arranged so as to have the handpiece in its field of view, and

a processing system for processing the images provided by the camera in order to locate the handpiece in relation to the part of the body,

a control system for controlling the emission of the light pulses, configured so as to automatically trigger the pulses on the basis of the movement determined from the information provided by the image processing system.

Such a device for treating the part of the body makes it possible to determine the temperature of the part of the body after it has received a light pulse. It also allows the treatment to be traceable and safeguards the operator.

The device may comprise one or a combination of the following features:

The treatment is non-invasive. The treatment is preferably non-surgical.

The treatment is esthetic.

The part of the body to be treated is an area of the surface of the skin.

The processing system for processing the images provided by the camera or cameras locates the handpiece with respect to the part of the body independently of the emission of the light pulses.

The light pulse control system is configured so as to automatically trigger the pulses on the basis at least of the position of the handpiece in relation to the part of the body located by the processing system for processing the images provided by the camera.

The handpiece comprises a marking making it easier for the camera or cameras to recognize.

The device comprises at least two cameras arranged so as to have the handpiece in its field of view.

The camera or cameras have a field of view that encompasses the entire part of the body to be treated.

The camera or cameras are fixed in relation to the body being treated.

The device comprises at least two cameras oriented in different viewing directions with respect to the part of the body to be treated.

The camera or cameras are arranged on the opposite side of the part of the body from the operator.

The device comprises a determination system for determining the movements of the part of the body in order to make it possible to take into account the movements of the part of the body during the treatment, in particular in the precise determination of the position of the handpiece.

The movement determination system may comprise a visual reference on the skin, for example a boundary of the part of the body to be treated, or better still a marking, in particular a grid on the part of the body to be treated, spaced geometric shapes, in particular distributed regularly over the part of the body to be treated, for example dots, lines, crosses or any other reference marking, the processing system for processing the images from the camera or cameras determining the movements of the body on the basis of the deformations of the grid or of the movements of the shapes with respect to one another that it detects.

As a variant, the movement determination system comprises projecting fringes onto the part of the body, the camera or cameras making it possible to analyze the fringes projected onto the skin and to deduce therefrom the movements of the part of the body.

The control system for controlling the emission of the light pulses is configured so as to automatically trigger the light pulses during the movement of the handpiece at a rate determined on the basis at least of at least one item of information provided by the camera or cameras.

The handpiece may comprise a motion detector, in particular an optical, capacitive or inertial motion detector.

The device may comprise a control system for controlling the emission of the light pulses, configured so as to automatically trigger the light pulses during the movement of the handpiece at a rate determined on the basis at least of at least one item of information provided by the motion detector.

One or a combination of the features described with reference to the device according to the first aspect of the invention.

Another subject of the invention, according to this fourth aspect of the invention, is a treatment method in accordance with the treatment method of the first aspect of the invention.

Another subject of the invention, according to a fifth of its aspects, is a device for treating a part of a human body by emitting light pulses, comprising

a handpiece for applying the light pulses to the part of the body, the handpiece being able to move in relation to the body and comprising

a window for outputting the light pulses onto a localized area of the part of the body,

a settable focusing system for varying the focal length of the light pulse at the output of the handpiece, and

a detector configured so as to detect the focus of the focusing system,

a computer configured so as to adapt at least one feature of the light pulses on the basis at least of an item of information provided by the detector.

The device may comprise one or a combination of the following features:

The handpiece may comprise a motion detector, in particular an optical, capacitive or inertial motion detector.

The device may comprise a control system for controlling the emission of the light pulses, configured so as to automatically trigger the light pulses during the movement of the handpiece at a rate determined on the basis at least of at least one item of information provided by the motion detector.

One or a combination of the features described with reference to the device according to the first aspect of the invention.

Another subject of the invention, according to this fifth aspect of the invention, is a method for treating a part of a body by emitting light pulses using a device according to this fifth aspect, comprising the step of setting the focal length of the light pulses in order to modify the size of the beam at the output of the handpiece.

The method may comprise one or a combination of the following features:

The method may comprise a step of applying a gel to the skin of the part of the body to be treated and/or to the handpiece,

The method may comprise a step of applying the handpiece to the part of the body to be treated and a step of scanning the part of the body to be treated with the handpiece, in particular while keeping the handpiece pressed on the skin.

The control system may automatically trigger the light pulses when the handpiece moves over the skin.

One or a combination of the features described with reference to the method according to the first aspect of the invention.

Another subject of the invention, according to a sixth of its aspects, is a device for treating a part of a human body by emitting light pulses, comprising:

a handpiece for applying the light pulses to the part of the body, the handpiece being able to move in relation to the body and comprising a window for outputting the light pulses onto a localized area of the part of the body, and

a system for determining the movements of the part of the body, providing information relating to the movements of the part of the body during the treatment, and

at least one camera arranged so as to have the handpiece in its field of view and/or a temperature detector for monitoring the local temperature of the area exposed to the light pulse, and a processing system for locating the handpiece in relation to the part of the body and/or determining the treated areas from the information provided by the camera and/or the temperature detector and at least one item of information provided by the determination system for determining the movements of the part of the body.

The device may comprise one or a combination of the following features:

The handpiece may comprise a motion detector, in particular an optical, capacitive or inertial motion detector.

The device may comprise a control system for controlling the emission of the light pulses, configured so as to automatically trigger the light pulses during the movement of the handpiece at a rate determined on the basis at least of at least one item of information provided by the motion detector.

One or a combination of the features described with reference to the device according to the first aspect of the invention.

Another subject of the invention, according to this sixth aspect of the invention, is a method for treating a part of a body by emitting light pulses using a device according to this sixth aspect, comprising the step of locating the handpiece with respect to the part of the body and/or determining the treated areas from the information provided by the camera and/or the temperature detector and at least one item of information provided by the determination system for determining the movements of the part of the body.

The method may comprise one or a combination of the following features:

The method may comprise a step of applying a gel to the skin of the part of the body to be treated and/or to the handpiece,

The method may comprise a step of applying the handpiece to the part of the body to be treated and a step of scanning the part of the body to be treated with the handpiece, in particular while keeping the handpiece pressed on the skin.

The control system may automatically trigger the light pulses when the handpiece moves over the skin.

One or a combination of the features described with reference to the method according to the first aspect of the invention.

Another subject of the invention, according to a seventh of its aspects, is a device for treating a part of a human body by emitting laser pulses, comprising

a base station comprising a laser source emitting laser pulses,

a handpiece for applying the laser pulses from the laser source to the part of the body, the handpiece being connected to the base station by an optical guide, the handpiece being able to move in relation to the body and comprising a window for outputting the light pulses onto a localized area of the part of the body,

the laser source having at least one row of laser diodes connected in series, comprising a plurality of laser diodes emitting at a wavelength in the non-visible domain and at least one laser diode emitting at a wavelength in the visible domain.

The device may comprise one or a combination of the following features:

The handpiece may comprise a motion detector, in particular an optical, capacitive or inertial motion detector.

The device may comprise a control system for controlling the emission of the light pulses, configured so as to automatically trigger the light pulses during the movement of the handpiece at a rate determined on the basis at least of at least one item of information provided by the motion detector.

One or a combination of the features described with reference to the device according to the first aspect of the invention.

Another subject of the invention, according to this seventh aspect of the invention, is a method for treating a part of a body by emitting light pulses using a device according to this seventh aspect.

The method may comprise one or a combination of the following features:

The method may comprise a step of applying a gel to the skin of the part of the body to be treated and/or to the handpiece,

The method may comprise a step of applying the handpiece to the part of the body to be treated and a step of scanning the part of the body to be treated with the handpiece, in particular while keeping the handpiece pressed on the skin.

The control system may automatically trigger the light pulses when the handpiece moves over the skin.

One or a combination of the features described with reference to the method according to the first aspect of the invention.

The invention will be able to be better understood on reading the following description of non-limiting exemplary implementations thereof, as well as on examining the appended drawing, in which:

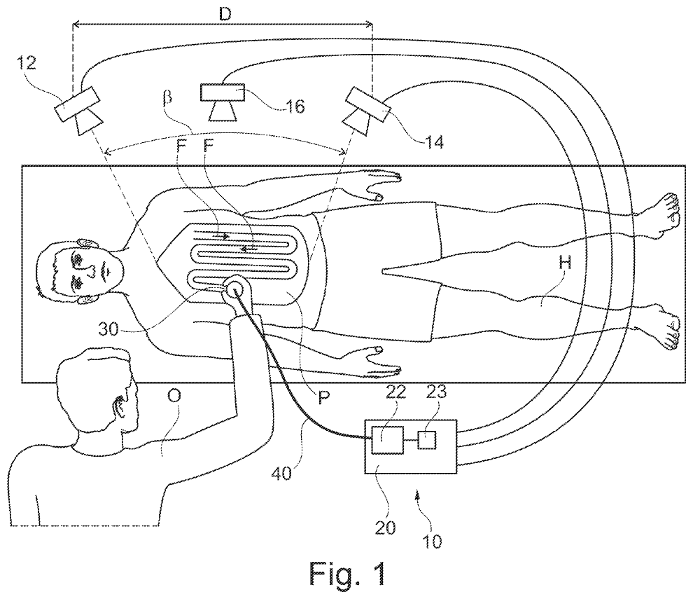

FIG. 1 schematically illustrates a treatment session using a device according to the invention, seen from above,

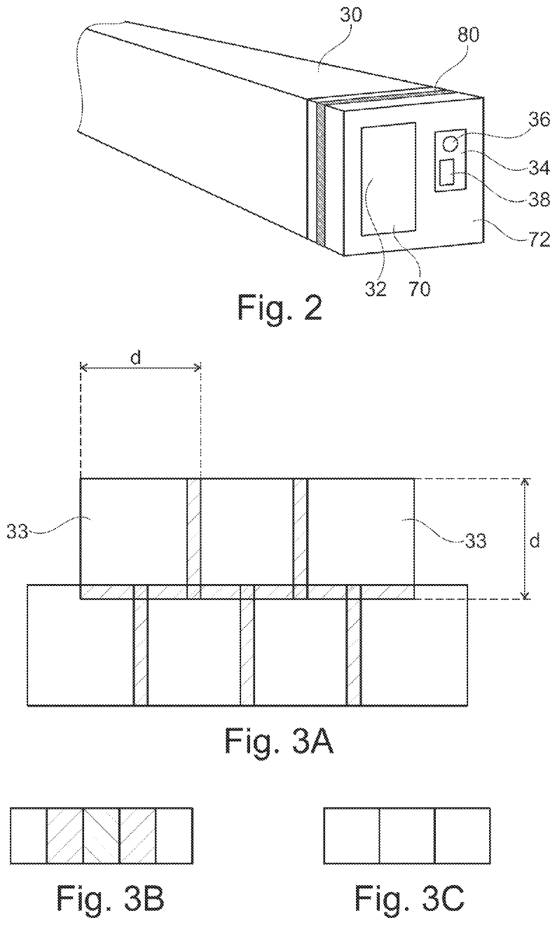

FIG. 2 is a schematic perspective depiction of the handpiece of the device from FIG. 1,

FIGS. 3A to 3C illustrate the arrangement of the areas that have received successive light pulses during the treatment session from FIG. 1,

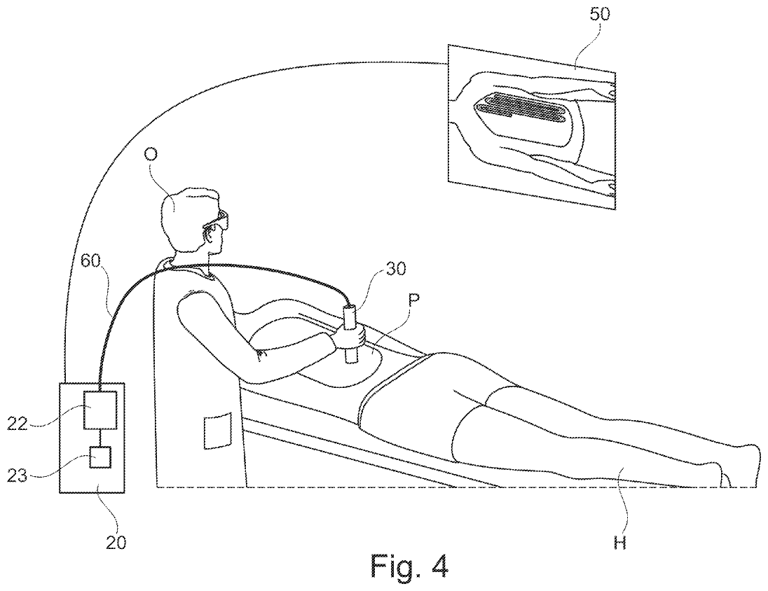

FIG. 4 shows the treatment session from FIG. 1 in another view,

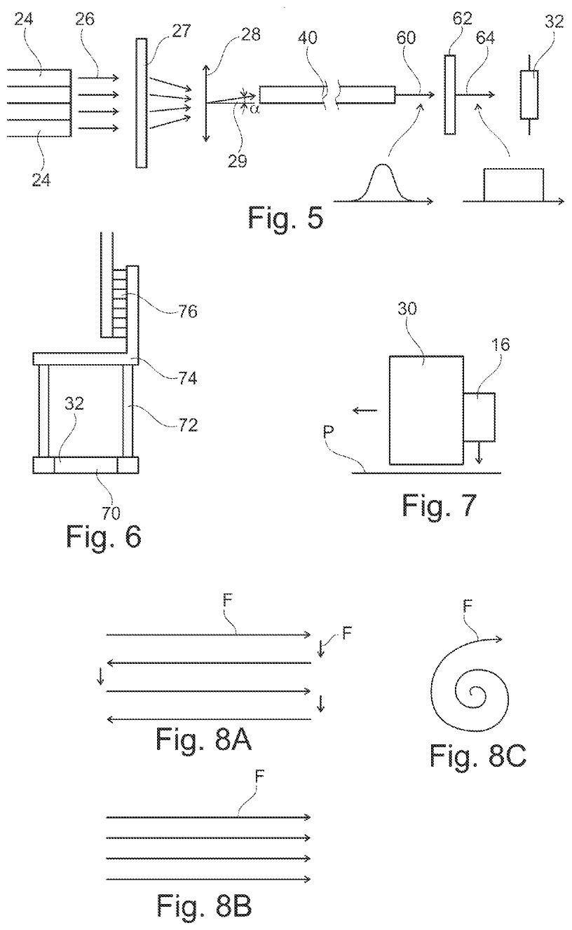

FIG. 5 schematically illustrates one example of an optical system of a device according to the invention,

FIG. 6 is a schematic sectional view of a handpiece of a device according to the invention,

FIG. 7 schematically shows a variant of a handpiece according to the invention,

FIGS. 8A and 8B show variants of scanning the part of the body to be treated with a device according to the invention,

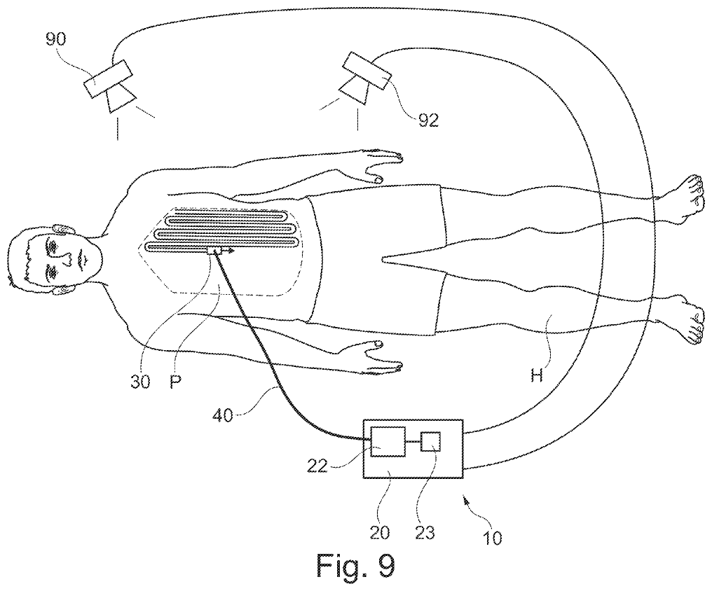

FIG. 9 schematically illustrates the use of a viewing system on the human body for viewing the treatment to be performed,

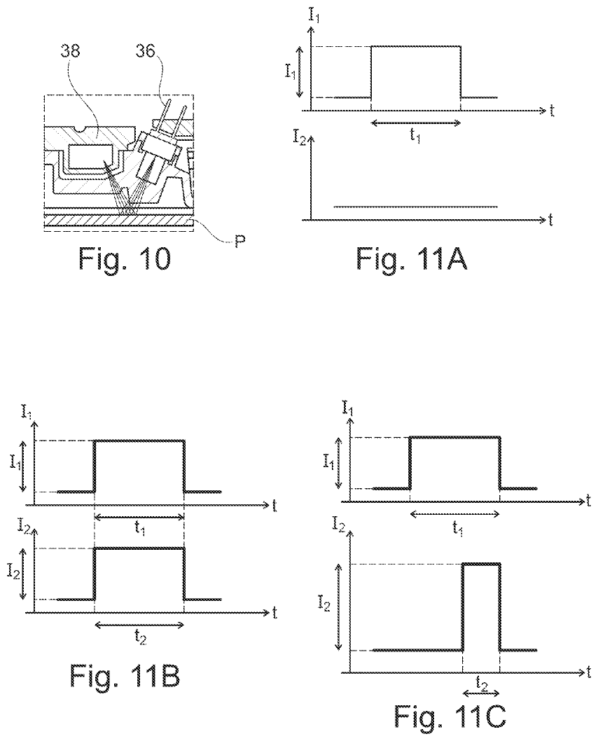

FIG. 10 schematically shows one example of a motion detector,

FIGS. 11A to 11C show trigger sequences for the rows of laser diodes of a device according to the invention,

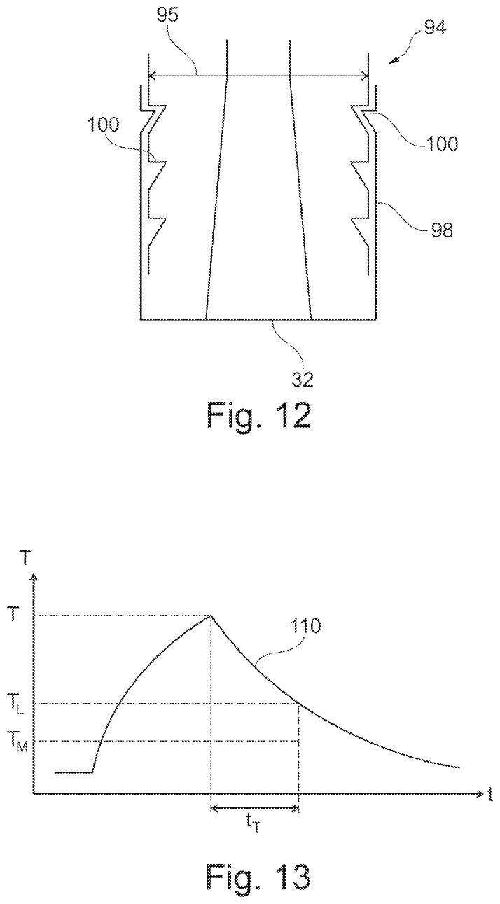

FIG. 12 is a schematic depiction of a settable focusing system of a device according to the invention, and

FIG. 13 is an example of a limit temperature decrease curve of the skin.

The treatment session shown in FIG. 1 is a hair removal session performed by an operator O on a part P to be treated of a human body H using a device 10 comprising a base station 20, a handpiece 30 able to move over the body H, and three cameras 12, 14 and 16 each connected to the base station 20. The camera 16 is a thermal camera.

The base station 20 comprises a laser source 22 and a cooling system, not shown, for cooling the laser source.

The handpiece 30 is connected to the base station by at least one flexible optical guide 40 allowing the laser pulses emitted by the laser source 22 to be transported to the handpiece 30.

The fact that the laser source 22 is remote from the handpiece 30 makes it possible to overcome the constraints in terms of size or weight of the cooling system, thereby making it possible to have a cooling system for cooling the laser source 22 that is more efficient than if it were in the handpiece 30. This also makes it possible to have a lighter and therefore more manageable handpiece.

As illustrated in FIG. 5, the laser source 22 comprises a plurality of laser diodes arranged in several rows 24, for example laser diodes arranged in 4 rows of 10 to 30 laser diodes each. The laser diodes in a row are connected in series, and the rows are either connected in parallel with one another or controlled independently of one another.

In each row 24, at least one of the laser diodes emits in the visible at a wavelength visible to the operator O during the treatment, and the other diodes all emit either at 750 nm or at 1064 nm. In this way, the components at 750 nm or 1064 nm of each row 24 make it possible to remove hair, and the component in the visible allows the laser pulses to be visible, thereby making it easier for the operator O to monitor the hair removal and increasing safety for said operator. The base part has at least one row 24a emitting at 750 nm and at least one row 24b emitting at 1064 nm. It is possible to vary the intensity of the laser pulses emitted by a row 24 by varying the supply current thereto. This allows a great deal of treatment modularity depending on the features of the body to be treated by making it possible to choose at the same time the rows 24a and/or 24b to be triggered, their intensities I₁ and/or 1₂ and the trigger sequence of each of the rows 24a or 24b. Specifically, the wavelength at 750 nm is well suited to hair removal for light phototypes, in particular I to IV, and the wavelength at 1064 nm is well suited to hair removal for dark phototypes, in particular V and VI.

For example, it is possible to trigger a row 24a or 24b at a certain intensity I₁ or I₂ and for a certain duration t₁ or t₂, as illustrated in FIG. 11A, or to simultaneously trigger different rows 24a and/or 24b at the same or a different intensity I₁ and I₂, respectively, and for the same duration t₁ and t₂, respectively, as illustrated in FIG. 11B, or it is possible to trigger one or more rows 24a emitting at 750 nm at an intensity I₁ over a first duration ti and to trigger one or more rows 24b emitting at 1064 nm with a greater intensity I₂ but over a second, shorter, duration t₂, the row or rows 24b being triggered such that their laser emission ends at the same time as the laser pulse emitted by the rows 24a, as illustrated in FIG. 11C. The beams emitted by the laser diodes 26 are concentrated by a lens 27 comprising a plurality of microlenses formed on a support, the microlenses being configured so as to make the beams from the laser diodes 26 converge on a lens 28. Said lens is arranged so as to introduce the resulting beam 29 into the optical guide 40 at an appropriate angle of incidence.

The laser pulses entering the optical guide preferably have a duration of between 1 ms and 100 ms, better still between 5 ms and 20 ms.

The optical guide 40 is preferably a liquid optical guide. The optical guide 40 is configured so as to allow laser radiation having an intensity of several kW to pass in continuous mode. As a variant, the optical guide is an optical fiber or a bundle of optical fibers.

As illustrated in FIG. 2, the handpiece 30 comprises a laser pulse output window 32, with a polygonal cross section, in particular a square or rectangular cross section, having a sapphire 70 that is preferably cooled. The output window 32 is preferably completely liquidtight. The output window 32 is surrounded by a metal sole 72, intended to come into contact with the skin during the treatment. The polygonal, in particular square shape of the output window makes it possible to optimize the number of flashes and to avoid an excessive overlap between flashes.

At the output of the optical guide 40, the handpiece 30 comprises an optical system 62 for transforming the cross section of the beam, which is generally substantially circular at the output of the optical guide 40, into a cross section of the emitted beam having a substantially rectangular shape without there being any loss of energy, preferably of substantially square shape, as illustrated in FIG. 3. The optical system 62 may also make it possible to homogenize the distribution of the light in the beam by transforming the Gaussian distribution of the light into a square distribution. The distribution of the light in the beam at the output of the optical system 64 is thus homogeneous over its entire cross section. This makes it possible to limit the risks of localized skin burns.

The corresponding optical system 64 comprises a lens having a plurality of microlenses formed on a support making it possible to transform the cross section of the beam, which is generally substantially circular at the output of the optical guide 40, into a cross section of the emitted beam having a substantially rectangular shape without there being any loss of energy, and a crystal configured so as to homogenize the distribution of the light in the beam.

Such an optical system allows a laser pulse to cover a localized area 33 of the body of the same shape as the output window 32, that is to say of polygonal shape, in particular square or rectangular shape, thereby making it possible, by moving the handpiece 30 over the body H, to easily juxtapose two areas 33 without there being any space between the two adjacent areas. The two adjacent areas 33 are contiguous and may overlap at least partially, as illustrated in FIGS. 3A or 3B, or not overlap, as illustrated in FIG. 3C. This allows a more effective treatment by limiting the risk of certain areas not being treated. The invention is not limited to a square output cross section of the laser pulses. What is important is that the output cross section of the laser pulses has two opposite parallel edges, making it possible to divide the part P of the body into a grid of tiles without any space between the adjacent areas 33.

The two adjacent areas 33 may overlap over 50% of their width d, as illustrated in FIG. 3A. In this way, each area of the skin receives two successive laser pulses. In the variant illustrated in FIG. 3B, the two adjacent areas overlap over a small portion of their width d, for example less than or equal to 20%, better still less than or equal to 10%. This slight overlap makes it possible to avoid certain areas of the skin not being fully treated. As a variant, as illustrated in FIG. 3C, the two adjacent zones are juxtaposed without overlapping.

As illustrated in FIG. 6, the sapphire 70 may be cooled by a cooling system comprising heat pipes 72 connecting the sole 72 of the handpiece comprising the sapphire 70 to a part 74 made from a material with good thermal conductivity, for example made from aluminum, itself in contact with Peltier-effect cells 76.

In one variant, not illustrated, the sapphire 70 is cooled only by the heat pipes connected to a metal part connected to a coolant, in particular water, or the sole 72 of the handpiece bearing the sapphire is made from a material with good thermal conductivity, in particular aluminum, connected directly, without heat pipes, to Peltier-effect cells.

At the output of the handpiece 30, each laser pulse has a maximum power flux density greater than or equal to 1 kW/cm². Such power allows good effectiveness of the body hair removal, even on fine hairs.

Depending on the properties of the skin, and in particular the phototype of the skin, the operator may modulate the energy and the power of the laser pulses before the treatment or during the treatment. This avoids burning the skin.

The handpiece may also comprise a settable focusing system 94 shown in FIG. 12 for modifying the focus of the laser pulse at the output of the handpiece 30. Such a system allows the operator O to concentrate the laser beam to a greater or lesser extent in a very precise area. The focusing system 94 is formed of a divergent lens 95 making the beam of the laser pulse conical, and of a telescopic portion 98 of the handpiece 30 for changing the distance between the lens 95 and the output window 32. Thus, by extending the telescopic portion 98 of the handpiece, the operator O is able to enlarge the cross section of the beam, or vice versa. The telescopic portion 98 of the handpiece comprises latching reliefs and corresponding reliefs 100 for blocking the telescopic portion in certain predefined extensions. The handpiece 30 comprises a detector, not shown, for detecting the extension of the telescopic portion 98. The device may then detect the predefined extension of the handpiece in which the handpiece is set, deduce the focus therefrom and automatically change the parameters of the light pulses according to the specific light pulse parameters associated with the predefined extension chosen by the operator. Such settable focusing makes it possible to set the surface dimension of the treated surface and the power of the light pulses on the basis of at least one feature of the area to be treated. For example, in the case of hairs with a large diameter, the operator may choose to extend the handpiece 30 in order to widen the beam width and treat a wide area with each laser pulse. Specifically, in the case of hairs with a large diameter, hair removal is effective with a lower power flux density than in the case of fine hairs. The wider the cross section of the beam at output, the faster the treatment.

As illustrated in FIG. 2, the handpiece 30 also comprises an optical or inertial motion detector 34, making it possible to detect the movements of the handpiece 30 in at least two directions. The motion detector 34 is fixed in relation to the handpiece. The motion detector 34 preferably comprises a light source 36, such as an LED or a laser diode, and a multi-pixel optical detector 38, for example a micro-camera, for detecting light coming from the light source 36 and reflected by the skin, as illustrated in FIG. 10. Analyzing the light received by the optical detector 38 makes it possible to deduce information on the movement of the handpiece 30 over the skin when said handpiece is moved while remaining in contact with the skin. It is then possible to automatically trigger the successive laser pulses on the basis of the movements of the handpiece 30 over the skin, as will be described below. The treated areas may therefore be distributed regularly over the part of the body to be treated.

The optical detector 38 preferably has a resolution greater than or equal to 100 pixels, better still greater than or equal to 250 pixels, even better still greater than or equal to 500 pixels, for example equal to 900 pixels. The optical detector 38 preferably takes more than 1000 images/s, better still more than 1500 images/s, even better still more than 3000 images/s, for example 6400 images/s.

The optical detector 38 preferably takes an image of the skin over a duration of less than or equal to 500 μs, better still less than or equal to 300 μs.

The pixels of the camera preferably each image an area of the skin with a width of less than or equal to 100 μm, better still less than or equal to 50 μm, even better still less than or equal to 20 μm. Such a resolution makes it possible, by analyzing the images taken by the optical detector 38, to determine the diameter of the hairs. The light source 36 may illuminate the skin continuously. As a variant, it illuminates the skin discontinuously with a very brief illumination duration. The illumination may be stroboscopic and of duration less than 300 μs, better still less than 100 μs.

Such a motion detector 34 makes it possible to detect a movement of less than or equal to 1 mm, better still less than or equal to 0.8 mm, even better still less than or equal to 0.5 mm.

The optical detector 38 may be a micro-camera that makes it possible to form an image of the skin, similar to the detectors fitted to computer mice.

The device comprises a processing system for processing the images provided by the optical detector 38. Such a system is able to detect hairs in said images. It may then determine, with knowledge of the position of the handpiece on the skin, the location and the diameter of each hair imaged and/or the number of hairs. From this information, it is possible to estimate the number of hairs on the part of the body to be treated and/or their associated diameters and/or the distribution of the hairs on the part of the body. This determination may be performed even if the surface detected by the micro-camera 38 is smaller than the surface dimension of the window 32. Specifically, in this case, it is possible to extrapolate the distribution of the hairs over the entire part of the body from the distribution of the hairs determined on the portion of the body imaged by the micro-camera 38. This makes it possible to adapt the parameters of the treatment on the basis of the density of hairs on the area to be treated. This estimation may be made independently of the emission of the laser pulses by the handpiece. It may then be carried out during the treatment, during scanning of the skin with the handpiece 30, or independently of the treatment by scanning the skin with the handpiece 30 without emitting the laser pulses.

As long as the handpiece 30 remains in contact with the skin, the motion detector 34 may make it possible to ascertain the position of the handpiece 30 in relation to its initial position with precision of the order of a millimeter.

Such a motion detector 34 also makes it possible to provide information relating to whether or not the handpiece 30 is being pressed on the skin. Specifically, when the handpiece 30 is no longer in contact with the skin, the optical detector 38 no longer makes it possible to receive a crisp image of the skin. The emission of the laser pulses may be stopped automatically when the handpiece 30 is not in contact with the skin. A visual or audible warning device may also be provided, for example a small diode or an information window on a screen 50, making it possible to signal to the operator O that the handpiece is no longer in contact with the skin and that no laser pulse is being emitted.

In one variant, not illustrated, the motion detector 34 comprises three light sources 36 of different colors, in particular red, green and blue. The light sources successively emit light. Each emission is detected by the optical detector 38 and a color image of the skin is recomposed from the images taken by the optical detector 38 for each of the wavelengths. The light sources may be three laser diodes or three LEDs. As a variant, the three light sources are integrated into a single laser diode or a single LED.

In these cases where a color image is formed, the motion detector 34 is preferably positioned upstream of the laser pulse output window 32 with respect to the direction of movement of the handpiece so as to make it possible to obtain a color image of the skin before it is subjected to a laser pulse. The features of the light pulses may then be changed automatically depending on the local features of the skin. For example, the device may automatically change the features of the laser pulses when the handpiece 30 changes from a tanned area to an untanned area. Such a variant requires the handpiece always to be oriented in the same way when moving the handpiece over the skin.

As another variant, the motion detector 34 may be a multi-pixel capacitive detector for imaging the microreliefs of the skin or an accelerometer.

The maximum firing rate frequency of the laser pulses is preferably greater than or equal to 30 Hz, better still 50 Hz. Such maximum frequencies in combination with automatic triggering of the laser pulses make it possible to quickly treat the part of the body to be treated.

The cameras 12 and 14 are electrically connected to the base station and are arranged so as to each observe the entire part P of the body to be treated. The two cameras 12 and 14 are arranged in two different orientations so as to have different viewing directions. They make it possible to view the part of the body and to locate the handpiece 30 thereon. They form with one another an angle β of between 45° and 120°, for example 90°. A processing system for processing the images provided by the cameras 12 and 14 makes it possible to have a three-dimensional view of the part of the body to be treated. This is particularly useful when treating a part of the body such as the arms or legs.

As illustrated in FIG. 2, the handpiece 30 may comprise a marking 80 allowing it to be identified by the cameras 12 and 14. This marking 80 preferably extends in at least two directions so as to allow the handpiece 30 to be recognized by the two cameras 12 and 14. The marking 80 may be a visual marking, for example a black line, or one or more light marks on the handpiece 30, for example a light halo or LEDs. It is then possible to determine the position of the handpiece 30 on the part of the body to be treated with precision of the order of a few millimeters. Such tracking of the handpiece 30 in relation to the part of the body to be treated makes it possible in particular to monitor the treatment in all circumstances, in particular when the operator has lifted the handpiece 30 from the skin or in the absence of detection of movement by the motion detector. Specifically, as soon as the operator lifts the handpiece 30, the motion detector 34 no longer makes it possible to monitor the movements of the handpiece 30 on the skin. The cameras 12 and 14 make it possible to take over the task of locating the handpiece.

To refine the determination of the positioning of the handpiece 30 on the part of the body, the information provided by the cameras 12 and 14 may be coupled with a determination system for determining the movements of the part P of the body in order to make it possible to take into account the movements of the body during hair removal, in particular in the precise determination of the position of the handpiece 30 and the determination of the areas of the body treated by the handpiece 30. The movement determination system may be a three-dimensional digital model of the human body to be calibrated on the basis of the body P to be treated. Such a model makes it possible, on the basis of the information provided by the cameras 12 and 14, to easily calculate the movements of the body without having to analyze the images from the cameras pixel by pixel, these being pre-calculated in the model.

As a variant, the movement determination system comprises a visual reference on the skin, for example a boundary of the part of the body to be treated, or better still a marking, in particular a grid on the part of the body to be treated, spaced geometric shapes, in particular distributed regularly over the part of the body to be treated, for example dots, lines, crosses or any other reference marking. The marking may be printed in ink, preferably allowing the wavelength or wavelengths of the light pulses to pass, in particular fluorescent and transparent to infrared. The processing system for processing the images from the camera or cameras may then determine the movements of the body on the basis of the deformations of the grid or of the movements of the shapes with respect to one another that it detects.

As another variant, the determination system for determining the movements of the part of the body comprises projecting fringes onto the part of the body, in particular using the projection system described above and at least one camera, in particular the camera or cameras for detecting the handpiece on the part of the body, or an additional projector, in particular a laser projector and/or an additional camera. Analyzing the deformations of the fringes then makes it possible to deduce the movements of the part of the body.

The determination system for determining the movements of the part of the body analyzes the movements of the part of the body every t milliseconds, t being less than or equal to 20 ms, for example equal to 15 ms.

All of the information provided by the motion detector 34 and the cameras 12 and 14 above is transmitted to a control system, not illustrated, configured so as to process this information and automatically trigger the laser pulses from the laser source 22 when the handpiece 30 moves in relation to the body H at a rate determined on the basis of the information provided by the motion detector. The rate is determined such that the distance covered by the handpiece between two laser pulses is less than or equal to d, where d is the dimension of the output window in the direction of movement of the handpiece, such that the areas subjected to the successive laser pulses overlap or do not overlap as illustrated in FIGS. 3A to 3C and described above. The laser pulses are triggered automatically, thereby limiting the risk of certain areas being overlooked and making it possible to treat the skin quickly by moving the handpiece over it without having to worry about triggering the pulses.

The thermal camera 16 is also electrically connected to the base station 10. It provides images of the temperature profile of the part to be treated. The images provided by the thermal camera 16 make it possible to deduce the local temperature of the area exposed to the laser pulse as soon as said area is no longer hidden by the handpiece 30. The temperature measurement is therefore performed a certain time t_{T} after the emission of the corresponding light pulse or pulses. This time t_{T} may be calculated from the speed of the handpiece 30 over the skin. The device then compares, as illustrated in FIG. 13, the measured temperature T_{M} with a limit temperature T_{L} measured on a limit decrease curve 110 at the time t_{T}, and if the measured temperature T_{M} is greater than the limit temperature T_{L}, the device stops the treatment or warns the operator O that the temperature of the skin is too high and that there is a risk of the skin burning. The limit temperature T_{L} should not in any case exceed 40° C.

By storing the information provided by the thermal camera 16 for each area of the body, the control system is able to distinguish between the areas of the part P to be treated that have already been treated and the as yet untreated areas. Specifically, the treated areas of the part of the body have, at a time in the treatment, experienced an increase in their temperature due to the light pulses that they have received and, during the treatment, the temperature detector has measured this temperature increase at least once.

In the variant illustrated in FIG. 7, the thermal camera 16 is arranged on the handpiece 30, in particular downstream of the output window 32, such that the thermal camera views the area of the skin that has immediately received a light pulse when the handpiece is moved. Such a variant requires the handpiece always to be oriented in the same way in relation to the direction of movement of the handpiece over the skin. In this case, it is preferable for the thermal camera 16 to be away from the skin.

From the data regarding the movements of the handpiece 30 as determined by the cameras 12 and 14 and/or by the motion detector 36 and/or the temperature information given by the thermal camera 16, the control system 23 is able to determine the number of passes of the handpiece 30 over each of the areas containing the part of the body. The control system 23 may then block the emission of a laser pulse when the handpiece 30 is arranged over an area over which it has already passed a predetermined number of times.

From the data regarding the movements of the handpiece 30 as determined by the cameras 12 and 14 and/or by the motion detector 36 and/or the temperature information given by the thermal camera 16, the control system 23 is able to determine the time since the last pass of the handpiece over a localized area of the part of the body. The control system 23 may then block the emission of a laser pulse when the time between two passes of the handpiece over the same area is not sufficient, in particular in order to allow the skin to have a temperature low enough to prevent it from being burned by the laser pulses. This time is predetermined and may be dependent on at least one feature of the skin, in particular its phototype.

The number of passes per unit area and the number of pulses per pass is determined by the operator prior to the treatment on the basis of the features of the laser pulses and of the skin. As described above, the successive areas subjected to the laser pulses may not overlap or overlap only slightly, such that the areas of the skin are each subjected to only one laser pulse in each pass. As a variant, the successive areas may overlap, for example by half, such that the areas of the part to be treated each receive two laser pulses in each pass. The choice of one or the other of these configurations may depend on the sensitivity of the skin. The features of the hairs and the skin may require a plurality of passes of the handpiece over each of the areas. In this case, the various passes should be spaced by a time that allows a good decrease in the temperature of the skin.

As illustrated in FIG. 4, the control system may display, on the screen 50, an image of the part to be treated and distinguish, on this image, between the areas of the part P to be treated that have already experienced the predetermined number of laser pulses and the other areas of the part to be treated. The control system may also display a temperature history on the screen 50 so as to allow monitoring of the temperature of the skin during the treatment. The screen may be a touchscreen and/or be connected to a mouse and a keyboard in order to allow the operator to choose the treatment parameters at the start and/or during the treatment, in particular on the basis of the phototype of the skin and/or the skin's reaction to the laser pulses.

The control system 23 preferably comprises a computer for determining in particular the frequencies of the laser pulses, the intensities of the laser pulses and their power on the basis of the parameters chosen by the operator at the start of the treatment, of the width of the selected laser pulse beam, of the focal length of the handpiece, of the information received by the device during the treatment, of the measurements performed during the treatment, in particular of the measured temperature, and of the skin after receiving the laser pulses.

The handpiece 30 may comprise a pressure sensor, not shown, for measuring the pressure with which the handpiece 30 presses on the skin. The control system is able to trigger the laser pulses only when the pressure detected by the pressure sensor is greater than a predetermined non-zero pressure. There may also be provision for a visual warning device, for example a diode or an information window on the screen 50, to warn the operator that the handpiece is not sufficiently pressed on the skin. Pressing the handpiece 30 on the skin makes it possible to improve the treatment, in particular in the case of a hair removal treatment, by displacing the blood under the skin.

The motion detector may make it possible to detect the pressure exerted by the handpiece on the skin by detecting the color of the skin under the pressure of the handpiece. Specifically, when a pressure is applied to the skin, the skin changes color, in particular becomes whiter.

In the case of hair removal, this also makes it possible to remove the hairs, and thus to improve the effectiveness of the treatment.

As illustrated in FIG. 9, the device 10 may also comprise a visual information system for viewing the progress of the treatment directly on the part P of the body, for example a system for distinguishing directly, on the part of the body, between the areas that have already been treated and the as yet untreated areas. Such a system may for example comprise two light projectors 90 and 92 projecting light onto the areas that have already been treated or, by contrast, onto the as yet untreated areas. Such a system may also distinguish between the number of passes that each area of the part of the body has experienced, for example through various colors according to the number of passes experienced. For example, the area to be treated is displayed in green by projecting a corresponding image on the body. In the first pass, the treated area appears in orange, and in red in the second one.

The device may also comprise a recording device for recording the progress of the treatment. Such a recording device may make it possible to record the image displayed by the screen during the treatment.

Some information taken by the device, in particular the history of the light pulses or the passes of the handpiece, the number of hairs treated and/or the cumulative temperature of the various areas, may be printed on a sheet in order to summarize the hair removal session.

The device may comprise or be connected to a client database for retaining information relating to the treatment from one session to the next, in particular the distribution of the hairs on the part of the body and the number of hairs measured. Thus, over the course of the sessions, the operator O is able to determine the history of the sessions and have an estimate of the effectiveness of the treatment, in particular by comparing the number of hairs and/or the distribution of the hairs on the part of the body from one session to another.

The device may also comprise a stop system actuated by the person receiving the treatment in order to stop the treatment in the event of experiencing pain. Such a system may constitute additional safety. The operator may stop the treatment whenever he wants.

The device may also comprise an operating mode selector that is able to adopt three different operating modes, an automatic mode in which the pulses are triggered automatically, a manual mode in which the pulses are triggered manually by the operator O, for example using a trigger on the handpiece 30, and a neutral mode without triggering laser pulses.

The handpiece 30 preferably does not have any rollers. The handpiece 30 slides over the skin by virtue of a gel being applied thereto. Applying a gel makes it possible to reduce the index differences between the interfaces passed through by the laser pulses, thereby improving the transmission of energy to the skin. The presence of the gel also makes it possible to avoid any release of smoke linked to the destruction of the hairs.

A description will now be given of a method for the treatment, by an operator O, of the part P of the body to be treated using a device according to the invention.

First of all, the operator selects the information relating to the client in the case of a client registered on the database or enters the information relating to the client in the case of a new client, and then determines one or more of the following elements:

the part of the body to be treated. He may for example define the part of the body to be treated on the image of the body displayed on the screen, or else define the region of the body to be treated directly on the body of the individual, for example using a marker. The operator may also mask sensitive areas, such as moles, and cover them with an opaque white felt in order to protect them from the laser pulses.

The phototype of the skin. The operator O selects the phototype of the skin from among phototypes I to VI. The viewing system may indicate the color of each of the phototypes in order to help the operator O choose the most suitable phototype.

The number N of laser pulses per unit area during one pass.

The number p of passes over each unit area of the part of the body.

The focal length, by choosing the length of the handpiece.

The diameter and the color of the hairs from among a plurality of predefined colors.

The manual or automatic operating mode.

The computer then determines the features of the laser pulses on the basis of the information provided by the operator O. It determines in particular the ratio of the distance that the handpiece has to cover between two laser pulses to the dimension of the cross section of the beam in the direction of movement and the trigger sequences of the rows of laser diodes, in particular their respective intensities, their respective on durations and their respective trigger times for each laser pulse. The device may comprise a plurality of trigger sequences for the prerecorded rows such that the computer selects the most suitable prerecorded sequence on the basis of the information provided by the operator O.

The operator O then applies the gel to the skin or the handpiece 30.

As illustrated in FIGS. 1 and 8A, the operator O then applies the handpiece 30 to the skin.

In manual mode, the operator O positions the handpiece and presses the trigger in order to trigger a laser pulse, and then moves the handpiece 30 a certain distance over the skin and presses the trigger again in order to trigger a new laser pulse, and so on. This mode is used in particular for waxing small areas of the face, such as the philtrum.

In automatic mode, the operator O scans the part P of the body with the handpiece 30. He preferably moves the handpiece 30 along a first axis parallel to one of the sides of the laser pulse output window 32 in a first direction and over the entire dimension of the part of the body in this direction. He then moves the handpiece in a direction perpendicular to this main direction by a distance corresponding to the height of the output window, and then moves the handpiece along a second axis parallel to the first axis in a direction opposite the first direction, and so on. Such a movement allows good coverage of the part P to be treated. In this movement scheme, most of the treatment takes place without lifting the handpiece 30. Depending on the selected number of passes p, the operator O scans or does not scan the part P of the body again with the same movement. It is preferable for the operator to allow time for the skin to cool down between two consecutive passes. He may for example resume scanning without stopping if the areas treated at the start of treatment have had time to cool down during the scanning, or wait a few seconds before the following pass. The successive passes are spaced by at least 1 s, preferably at least 2 s, better still at least 3 s.

The handpiece 30 is oriented, during its movement, such that the main direction of movement is parallel to one of the sides of the laser pulse output window 32.

The operator O moves the handpiece without having to worry about triggering the laser pulses. These are triggered automatically by the device, and the operator is able to monitor the correct progress directly on the screen 50. In real time, he views the position of the handpiece 30 in relation to the areas that have already been treated directly on the screen 50. At the end of each pass, the operator O may move the handpiece 30 directly over the areas that have not experienced a laser pulse in this pass or go to the next pass, and the screen 50 allows him to identify the untreated areas and the number of passes made over each area.

In the event of an alert from the device linked for example to an excessively high temperature of the skin after the application of a laser pulse, to the loss of contact of the handpiece with the skin or to an excessively fast speed of movement of the handpiece, to a change in the features of the skin and/or to excessively little contact pressure of the handpiece on the skin, the operator may be warned in order to allow him to take an appropriate action, for example to modify the features of the laser pulses, reposition the handpiece on the skin, slow down the movement of the handpiece on the skin and/or exert more pressure on the skin with the handpiece. The alert may be output by activating one or more indicator lights, for example LEDs, or through an indication on the screen 50, for example the opening of a temporary window on the screen. Such an alert is accompanied, in the case of loss of contact of the handpiece with the skin and excessively low pressure, by stopping the emission of the laser pulses, and may or may not be accompanied by such a stoppage in the other three cases.

The operator O may decide to interrupt the treatment at any time by stopping the movements of the handpiece or by lifting the latter. The operator may resume the treatment at any time from where it was stopped by following the information on the screen 50. When the operator repositions the handpiece 30 on the skin, the device detects the resumption of the treatment and again triggers the laser pulses when the handpiece is positioned on a not fully treated area of the part of the body and this area is cold enough.

At the end of the treatment, the operator is able to view a treatment report on the screen, in particular the history of the laser pulses applied to each of the areas of the part of the body, the features of the applied laser pulses, the number of hairs treated, the distribution of the hairs over the part of the body, and/or the cumulative history of the body temperature over the entire treatment.

During the various treatment sessions, the operator may determine the effectiveness of the treatment by displaying the history of the distribution of the hairs, their associated diameters and/or the number of hairs on the part of the body to be treated on the screen.

In the case described above, the distribution of the hairs, the diameter of the hairs and/or the number of hairs are determined during the treatment. As an alternative, the operator may switch the device to neutral mode and scan the part of the body with the handpiece. No laser pulse is triggered in neutral mode. Such a mode may make it possible to estimate a map of the skin, and/or the distribution of the hairs on the part of the body and/or their associated diameters, and/or the number of hairs. This mode has the advantage of being able to scan a large part of the surface to be treated, in particular more than 50% of the surface.

The operator may move the handpiece 30 at a speed of less than or equal to 50 cm/s, preferably between 5 and 20 cm/s.

The operator O preferably wears protective glasses throughout the treatment in order to filter the light from the laser pulses and thus protect his eyes.

Such a treatment device and such a treatment method make it possible to treat a surface area of 1600 cm², for example a man's back, in less than 5 min, better still in less than 2 min, better still in less than 1 min.

In the variant illustrated in FIG. 8B, the operator O may move the handpiece 30 along parallel lines and always in the same direction. At the end of a line, the operator O lifts the handpiece 30 in order to reposition it at the start of the following line. In this movement scheme, it is necessary to lift the handpiece 30. This movement is particularly suitable if the motion detector 34 has three laser diodes of different colors and/or the thermal camera 16 is on the handpiece 30, as described above. This also makes it possible to treat a better-cooled area in each new scan. The cameras 12 and 14 make it possible to continuously ascertain the position of the handpiece 30 in relation to the body.

The invention is not limited to what has just been described with reference to the figures.

For example, the screen and the viewing system directly on the body may be replaced with a virtual reality or augmented reality headset.

The thermal camera may be replaced with a temperature sensor or skin color sensor.

The operator may be replaced with a robotic arm, possibly controlled directly by the device. An operator may then enter the information described above and the robotic arm performs the treatment. The operator may monitor the progress of the treatment on the screen and stop it at any time.

The above text describes a hair removal treatment. However, such a device may be used in the context of a photo-rejuvenation treatment or a vascular treatment, in particular at the expense of a few adjustments linked to the features of each of these treatments. The wavelengths of the laser diodes are preferably between 500 and 530 nm for a vascular treatment device.

The above text describes a laser radiation treatment device. However, the invention is not limited to such an example, and a PPL or IPL device may be used.

1. A device for treating, in particular for hair removal or photo-rejuvenation or vascular treatment, a part of a human body by emitting light pulses, comprising:
a handpiece for applying the light pulses to the part of the body, the handpiece being able to move in relation to the body and comprising:
a window for outputting the light pulses onto a localized area of the part of the body, and
an optical, capacitive or inertial motion detector,
a control system for controlling the emission of the light pulses, configured so as to automatically trigger the light pulses during the movement of the handpiece at a rate determined on the basis at least of at least one item of information provided by the motion detector.

2. The device as claimed in claim 1, wherein the motion detector makes it possible to determine the direction and the speed of movement of the handpiece over the part of the body.

3. The device as claimed in claim 1, wherein the rate is determined such that the distance covered by the handpiece between two light pulses is less than or equal to d, preferably less than d, where d is the dimension of the output window in the direction of movement of the handpiece.

4. The device as claimed in claim 1, wherein the maximum emission frequency of the light pulses is greater than or equal to 10 Hz, better still greater than or equal to 30 Hz, even better still greater than or equal to 50 Hz.

5. The device as claimed in claim 1, wherein the light pulses are laser pulses and the device comprises a base station comprising a laser source emitting laser pulses, the handpiece being optically connected to the laser source by at least one optical guide.

6. The device as claimed in claim 5, wherein the base station comprises a plurality of laser diodes arranged in a plurality of rows, the diodes in one and the same row of laser diodes being connected in series and the various rows of laser diodes being connected in parallel, or preferably controlled independently, each row of laser diodes having laser diodes all emitting either at around 750 nm or at around 1064 nm, and optionally one or more laser diodes emitting in the visible.

7. The device as claimed in claim 6, wherein the control system determines the trigger sequence of the laser diodes, in particular the rows of laser diodes to be triggered, the intensities of each of the rows of laser diodes, the trigger duration of each of the rows of laser diodes and/or the trigger time of each row of laser diodes, on the basis of at least one feature of the skin, in particular the phototype of the skin, the feature of the skin preferably being determined from the information provided by the motion detector and/or from data chosen by the operator.

8. The device as claimed in claim 1, wherein the motion detector is outside the light pulse output window, preferably adjacent to the light pulse output window.

9. The device as claimed in claim 1, wherein the motion detector is optical and preferably comprises a light source, in particular an LED or preferably a laser diode, and a multi-pixel optical detector, preferably a camera, configured so as to image a portion of the skin illuminated by the light source.

10. The device as claimed in claim 1, comprising a processing system for processing the images provided by the motion detector.

11. The device as claimed in claim 10, wherein the processing system for processing the images provided by the motion detector determines a feature of the skin, and the control system drives the light pulses on the basis of said feature of the skin.

12. The device as claimed in claim 10, wherein the treatment is hair removal and the processing system for processing the images provided by the motion detector detects the hairs in said images and determines the location on the skin and/or the diameter of each detected hair and/or the number of hairs detected.

13. The device as claimed in claim 10, wherein the processing system for processing the images provided by the motion detector detects pressing of the handpiece on the skin, the control system being configured so as to prevent a light pulse from being emitted when the handpiece is not being pressed against the skin.

14. The device as claimed in claim 1, wherein the handpiece comprises an optical system arranged upstream of the window, in particular between the optical guide and the window, for transforming the cross section of the beam before the window, in particular at the output of the optical guide, into an emitted beam cross section having, in a direction of movement of the handpiece, two substantially parallel opposing edges, in particular having a rectangular or square shape.

15. The device as claimed in claim 1, comprising a settable focusing system for varying the focal length of the laser pulse at the output of the handpiece, the handpiece preferably comprising a focusing detector configured so as to detect the focus of the focusing system, the device preferably comprising a computer configured so as to adapt at least one feature of the light pulses on the basis at least of an item of information provided by the focusing detection system.

16. The device as claimed claim 1, wherein the handpiece comprises a cooling system for cooling the handpiece, the cooling system for cooling the handpiece preferably comprising at least one Peltier-effect cell and/or at least one heat pipe.

17. The device as claimed in claim 1, wherein the temperature of the face of the handpiece configured so as to come into contact with the skin is, during operation, less than or equal to 20° C., preferably less than or equal to 15° C., better still less than or equal to 10° C., even better still less than or equal to 5° C.

18. The device as claimed in claim 1, wherein each laser pulse emitted by the output window has a power flux density greater than or equal to 200 W/cm², preferably greater than or equal to 1 kW/cm², better still greater than or equal to 2 kW/cm².

19. The device as claimed in claim 1, comprising at least one camera, preferably at least two cameras, each arranged so as to have the handpiece in its field of view, preferably a field of view that encompasses the entire part of the body to be treated, and a processing system for processing the images provided by the camera or cameras in order to locate the handpiece with respect to the part of the body from at least these images.

20. The device as claimed in claim 1, comprising a temperature detector, in particular a thermal camera, for monitoring the local temperature of the area exposed to the light pulse, the control system being configured so as to block triggering of the light pulses or to emit an alert signal for the operator when the local temperature is greater than a limit temperature, the limit temperature preferably being calculated on the basis of the speed of movement of the handpiece and of a predetermined temperature decrease curve depending on at least one feature of the skin, in particular the phototype of the skin.

21. The device as claimed in claim 1, comprising at least one visual information system for viewing the areas to be treated in relation to the as yet untreated areas of the part of the body to be treated, in particular at least one projection system for projecting information onto the part of the body in order to guide the operator during the treatment.

22. The device as claimed in claim 1, comprising a treatment viewing system, in particular a screen, configured so as to display an image of the part of the body, in particular a three-dimensional image, and optionally one or more of the following features:
the position of the handpiece on the body as provided by the camera or cameras,
the treated areas of the part of the body and/or the untreated areas of the part of the body, and/or
the history of the temperature of the skin based on the information provided by the temperature detector since the start of the treatment at each point of the part of the body to be treated.

23. The device as claimed in claim 1, wherein the device determines the as yet untreated areas of the part of the body, and the control system is configured so as to automatically trigger the emission of the light pulses when the handpiece is positioned on an as yet untreated area.

24. The device as claimed in claim 1, comprising a determination system for determining the movements of the part of the body in order to make it possible to take into account the movements of the part of the body during the treatment, in particular in the precise determination of the position of the handpiece and the determination of the areas of the body treated by the handpiece.

25. A method for treating, in particular for hair removal or for photo-rejuvenation, a part of the body using the device according to the invention, comprising the steps of
applying a gel to the skin of the part of the body to be treated or to the handpiece
applying the handpiece to the part of the body to be treated,
scanning the part of the body to be treated with the handpiece while keeping the handpiece pressed on the skin,
the control system automatically triggering the light pulses when the handpiece moves over the skin.

26. The method as claimed in claim 25, wherein the handpiece scans the part of the body to be treated at a speed of less than or equal to 50 cm/s, preferably between 1 and 50 cm/s.

27. The method as claimed in claim 25, comprising a step of the operator selecting certain parameters, in particular a step of selecting the number of light pulses per pass of the handpiece and the number of passes over each unit area of the part of the body to be treated.

28. The method as claimed in claim 25, wherein the successive passes over one and the same area of the part of the body are temporally spaced by a duration of at least 1 s, preferably at least 2 s, better still at least 3 s.

29. The method as claimed in claim 25, wherein the scanning of the handpiece over the part of the body is scanning along adjacent lines while always scanning in the same direction or in opposite directions from one line to another, preferably always scanning in the same direction.

30. The method as claimed in claim 25, wherein the handpiece is applied to the skin with a pressure greater than or equal to a predetermined pressure, in particular greater than or equal to 10 g/cm², preferably 20 g/cm².

31. The method as claimed in claim 25, wherein the treatment is a hair removal treatment and the method comprises a step of estimating the distribution of the hairs, their associated diameters and/or the number of hairs on the part of the body.
